(19) United States
(12) Patent Application Publication
(10) Pub. No.: US 2002/0058287 A1
(43) Pub. Date: May 16, 2002

(54) Novel small nuclear RNA vectors and uses therefor

(75) Inventors: David de Graaf, Cambridge, MA (US); Eric S. Lander, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 09/804,481

(22) Filed: Mar. 12, 2001

Related U.S. Application Data
(60) Provisional application No. 60/188,304, filed on Mar. 10, 2000.

(51) Int. Cl. G01N 33/53; G01N 33/567; C12N 15/00; C12N 5/06; C12N 15/87

(52) U.S. Cl. 435/7.1; 435/7.21; 435/325; 435/455; 435/320.1

(57) ABSTRACT

The present invention relates to the discovery of a recombinant vector into which preselected DNA modifications can be readily inserted. Digestion of the vector with a dual cleavage restriction enzyme forms insertion sites which allow the directed placement of an insertion cassette comprised of a double stranded modification fragment containing a preselected sequence modification linked to a pair of single-stranded overhangs with DNA sequences complementary to the DNA sequences of the insertion sites formed in the isolated DNA of the vector. Methods of producing recombinant vectors, and methods of utilizing the vectors to create cell libraries, to identify snRNAs which suppress expression of transcription products, to suppress expression of transcription products and to deliver antisense targeting sequences are also within the scope of the invention.

### RELATED APPLICATION

This application claims the benefit of U.S. Provisional Application No. 60/188,304 filed Mar. 10, 2000, the entire teachings of which are incorporated herein by reference.

### BACKGROUND OF THE INVENTION

Small nuclear ribonucleic acids (snRNAs) are essential components of small nuclear ribonucleoprotein complexes (snRNPs) which, when assembled with additional proteins, form the large ribonucleoprotein complex known as the splicesome. The splicesome is responsible for precursor mRNA splicing, the process that removes introns from RNA transcripts before protein production. An individual snRNA is generally about 250 nucleotides or less in size (Alberts, B. et al., “Molecular Biology of the Cell”, Third Edition, Garland Publishing, Inc., New York, 1994, 365-385). The various splicesome snRNAs have been designated as U1, U2, U3 . . . U12, due to the generous amounts of uridylic acid they contain (Mattaj, I. W. et al., 1993, FASEB J 15 7:47-53).

There has long been interest in utilizing the various splicing functions of individual U snRNAs to inhibit or modify transcription, and, thereby, to suppress undesired expression products (Cohen, J. B., etal., 1994, PNAS 91:10470-10474).

Such suppression has enormous therapeutic potential.

Moreover, because snRNA structure is highly similar to naturally occurring antisense RNAs, U snRNAs have been utilized in vectors constructed to deliver antisense targeting sequences. Dramatically reduced expression of fibrillin-1 using hybrid cRNAs that incorporate structural elements of U1 snRNA containing antisense targeting sequences has been reported (Montgomery, R. A., et al., 1997, Hum Mol Gen 6:519-525). Likewise, the expression of scatter factor/hepatocyte growth factor and its c-met receptor was suppressed after the transfection of transgenes containing U1 snRNA, a hammerhead ribozyme and antisense sequences into glioblastoma cells (Abounader, R., et al., 1999, J of NCI 91:1548-1556). The delivery of HIV-1 antisense sequences inserted into U1 snRNA was also found to produce immune cells stably resistant to HIV-1 (Liu, D, et al., 1997, J of Virology 71(5):4079-4085).

However, the further development of therapeutic uses for U snRNAs has been hampered by the difficulty of producing libraries containing sufficient numbers of modified snRNA sequences to evaluate and optimize the capabilities of individual modifications for inhibiting transcription. Site-directed mutagenesis has been used to generate some mutations of interest, but creating the large and diverse libraries required to fully exploit this technology using that method has proved difficult. Moreover, the vectors used to successfully deliver antisense targeting sequences, such as those described in the preceding paragraph, were constructed using methods requiring pairs of restriction enzymes. These methods are not only cumbersome, but generally result in the addition of extraneous nucleotides to the sequences being modified, which can prove detrimental.

Thus, there exists a general need for materials and methods which can efficiently deliver an array of suitable modifications of individual DNA sequences for evaluation and use. This need is particularly acute in the area of splicesome technology.

### SUMMARY OF THE INVENTION

As described in further detail herein, the invention relates to a recombinant vector comprising an isolated DNA sequence encoding an snRNA (e.g., U1), wherein the snRNA sequence has been modified to contain one or more restriction sites such that digestion with at least one restriction enzyme, and preferably only one restriction enzyme (e.g., Bae 1), allows easy insertion of target-specific sequences (inserts). In a preferred embodiment, the modification is such that the restriction enzyme(s) cleaves 3′ and 5′ of the region to be excised, thereby eliminating the problems associated with the insertion of additional (extraneous) nucleotides into the snRNA sequence. One advantage of such a vector is more efficient and faster cloning of the inserts, as well as the generation of libraries of snRNA molecules with altered specificity. Vectors of the invention target mRNA comprising a nucleotide sequence which is complementary to the target-specific sequence, thereby inhibiting splicing of the target mRNA and inhibiting expression of the transcription product of the mRNA. Alternatively, vectors of the invention can be used to deliver particular antisense sequences to a target mRNA, thereby inhibiting expression of the transcription product of the MRNA in a manner similar to traditional antisense methodologies.

Thus, in one embodiment, the present invention relates to the discovery of a recombinant vector in which preselected DNA modifications (e.g., insertion of one or more DNA sequences) can be readily made. According to the invention, a recombinant vector comprising a DNA sequence encoding a snRNA is modified to contain one or more restriction sites within the snRNA sequence such that digestion with at least one restriction enzyme, and preferably only one restriction enzyme, produces a double-stranded restriction fragment with single-stranded overhangs at each end. In a preferred embodiment, the restriction enzyme is a dual cleavage restriction enzyme. Excision of this restriction fragment from DNA contained in the vector forms insertion sites in the snRNA DNA of the vector; these insertion sites comprise single-stranded overhangs which are complementary to the single-stranded overhangs of the restriction fragment. These sites readily allow the directed placement of an insertion cassette comprising a double-stranded modification fragment containing a preselected DNA sequence. Each strand of the modification fragment is linked to a DNA sequence which is complementary to one of the single-stranded overhangs of the insertion sites in the vector. FIG. 4 illustrates the structure of a Bae1/U1 construct of the invention which is illustrative of the above description.

The vector permits the rapid and efficient creation of large libraries containing an array of preselected sequence modifications, so that optimally-performing sequences can be quickly detected and utilized. Methods of producing recombinant vectors, and methods of utilizing the vectors to create cell libraries, to identify snRNAs which suppress expression of transcription products, to suppress expression of transcription products and to deliver antisense targeting sequences are also within the scope of the invention.

In one aspect, the invention pertains to a recombinant vector containing isolated DNA encoding a snRNA in which the isolated DNA includes an insertion cassette contained between at least two insertion sites. In a preferred embodiment, the snRNA is selected from the group of snRNAs with splicing functions. In a particularly preferred embodiment, the snRNA is U1 snRNA. In a particularly preferred embodiment, the snRNA is U6 snRNA.

An insertion cassette of the invention can include a double-stranded DNA modification fragment containing any number of DNA base pairs. In embodiments, the insertion cassette includes a double-stranded DNA sequence of about 5, 10, 11, 12, 13, 14, 15, 16, 17, 18, 19, 20, 21, 22, 23, 24, 25, 26, 27, 28, 29, 30, 35, 40, 45, 50, 55 or 60 base pairs. In a preferred embodiment, the insertion cassette includes a double-stranded DNA sequence of about 30 base pairs. In a particularly preferred embodiment, the insertion cassette includes a double-stranded DNA sequence of 28 base pairs.

In another aspect, the invention pertains to a recombinant vector containing isolated DNA encoding a snRNA in which the isolated DNA includes a Bae 1 restriction fragment.

In a further embodiment, the vector includes two insertion sites formed by the excision of the Bae 1 restriction fragment from the isolated DNA of the vector. In preferred embodiments, the two insertion sites include DNA sequences which are the complements of SEQ ID NO: 2 and SEQ ID NO: 3.

In yet a further embodiment, the vector also includes an insertion cassette. In embodiments, the modification to the DNA sequence encoding the snRNA as contained within the modification fragment of the insertion cassette can result in an increase to the total number of nucleotides contained in the isolated DNA of a vector, a decrease to the total number of nucleotides contained in the isolated DNA of a vector, or, alternatively, the total number of nucleotides in the isolated DNA of the vector can remain unchanged. In a preferred embodiment, the insertion cassette comprises the same number of nucleotides, or about the same number of nucleotides, as contained in a recognition fragment produced by the Bae 1 restriction enzyme on a DNA encoding a U1 snRNA.

In a preferred embodiment, the insertion cassette includes a modification fragment containing a modification to the first 11 nucleotides of a U1 snRNA. In this embodiment, either a single nucleotide of the first 11 nucleotides of a U1 snRNA is modified, or a plurality of nucleotides of the first 11 nucleotides of a U1 snRNA are modified.

In embodiments, the insertion cassette includes a single-stranded overhang at each end of the modification fragment which consists of about 3, 4, 5, 6, 7, 8, 9 or 10 nucleotides. In a preferred embodiment, each overhang consists of about 5 nucleotides.

In an embodiment, each overhang can be located at a 3′ end of the modification fragment. In an alternate embodiment, each overhang can be located at a 5′ end of the modification fragment.

In a preferred embodiment, the insertion sites include single-stranded DNA sequences complementary to the single-stranded DNA sequences contained in a restriction fragment produced by the Bae 1 restriction enzyme on a DNA encoding a U1 snRNA. In a preferred embodiment, the insertion sites include single-stranded DNA sequences complementary to the single-stranded DNA sequences contained in an insertion cassette of the invention.

In another aspect, the invention pertains to a method of producing a recombinant vector including isolated DNA encoding a product of interest in which the isolated DNA includes an insertion cassette contained between at least two insertion sites including the steps of inserting isolated DNA encoding a product of interest into the vector; contacting the isolated DNA with a dual cleavage restriction enzyme that excises a restriction fragment including a double-stranded DNA modification fragment with a single-stranded DNA overhang at each end of the fragment; excising the restriction fragment from the isolated DNA of the vector so that at least two insertion sites are formed in the isolated DNA; obtaining an insertion cassette in which the DNA sequence of the single-stranded overhang linked to each end of the modification fragment is complementary to the insertion sites formed in the isolated DNA; and ligating the insertion cassette into the isolated DNA of the vector between the insertion sites formed by the excision of the restriction fragment, thereby producing a recombinant vector comprising an insertion cassette contained between at least two insertion sites.

In a preferred embodiment, the product of interest is a snRNA with a splicing function. In a particularly preferred embodiment, the snRNA is U1. In a particularly preferred embodiment, the snRNA is U6.

In a preferred embodiment, the insertion cassette includes a modification to the first 11 nucleotides of a U1 snRNA. In this embodiment, either a single nucleotide of the first 11 nucleotides of a U1 snRNA is modified, or a plurality of nucleotides of the first 11 nucleotides of a U1 snRNA are modified.

In a preferred embodiment, the dual cleavage restriction enzyme is Bae 1.

In embodiments, the insertion cassette includes a single-stranded overhang at each end of the modification fragment which consists of about 3, 4, 5, 6, 7, 8, 9 or 10 nucleotides. In a preferred embodiment, each overhang consists of about 5 nucleotides.

In an embodiment, each overhang can be located at a 3′ end of the modification fragment. In an alternate embodiment, each overhang can be located at a 5′ end of the modification fragment.

In a preferred embodiment, the insertion sites include single-stranded DNA sequences complementary to the single-stranded DNA sequences contained in a restriction fragment produced by the Bae 1 restriction enzyme on a DNA encoding a U1 snRNA. In a preferred embodiment, the insertion sites include single-stranded DNA sequences complementary to the single-stranded DNA sequences contained in an insertion cassette of the invention. In a preferred embodiment, each insertion site includes about 5 nucleotides.

In yet another aspect of the invention, a cell transformed by a recombinant vector comprising isolated DNA encoding a snRNA, in which the DNA includes an insertion cassette contained between at least two insertion sites is provided.

In embodiments, the cell can be procaryotic or eukaryotic. The cell can be bacterial, yeast or mammalian. In a preferred embodiment, the cell is a mammalian cell.

In another aspect, a cell library comprising cells transformed by a plurality of recombinant vectors including isolated DNA encoding a snRNA, wherein the DNA includes an insertion cassette contained between at least two insertion sites is provided. A cell library of the invention can include any number of cells transfected with vectors containing any number of insertion cassettes containing any number of different or unique modification fragments. In a preferred embodiment, a cell library includes insertion cassettes containing at least 5, 10, 15, 20, 25, 50, 75, 100 and 200 or more different modification fragments.

In another aspect, the invention pertains to a method of identifying a modification of a snRNA which suppresses transcription of a transcription product in a cell including the steps of determining a base level of transcription of a transcription product in a cell; producing at least 10 recombinant vectors comprising isolated DNA encoding a snRNA in which the isolated DNA of each of the recombinant vectors includes an insertion cassette containing a different modification fragment contained between at least two insertion sites of the vector; introducing each vector containing a modification into a cell, under conditions suitable for delivery of the snRNA into the cell; comparing the level of transcription of the transcription product in each cell including a vector containing the modified snRNA with the base level of transcription of the transcription product in the cell; and determining which snRNA modifications inhibit transcription in the cell, whereby, if the level of transcription of the transcription product in the cell including the vector containing the modified snRNA is less than the base level of transcription of the transcription product in the cell, a modification which suppresses expression of a transcription product in the cell has been identified.

In another aspect of the invention, a method of suppressing expression of a transcription product in a cell is provided including the steps of producing a recombinant vector containing isolated DNA encoding a snRNA, in which the isolated DNA includes an insertion cassette contained between at least two insertion sites; introducing the vector into the cell, under conditions suitable for delivery of the snRNA into the cell; and utilizing the snRNA to inhibit transcription in the cell, thereby, suppressing expression of a transcription product in the cell.

In yet another aspect of the invention, a method of delivering an antisense targeting sequence into a cell nucleus is provided including the steps of inserting an antisense targeting sequence into a recombinant vector containing an isolated DNA encoding a snRNA, in which the DNA includes an insertion cassette contained between at least two insertion sites; introducing the vector into the cell, under conditions suitable for delivery of the antisense targeting sequences across the cell membrane and into the cell nucleus, whereby the antisense targeting sequences are delivered to the cell nucleus.

### BRIEF DESCRIPTION OF THE DRAWINGS

The foregoing and other objects, features and advantages of the invention will be apparent from the following more particular description of preferred embodiments of the invention and as illustrated in the accompanying figures.

FIG. 1A-1E depicts a sequence of human U1 snRNA (SEQ ID NO: 1).

FIG. 2 depicts a schematic representation of a vector of the invention.

FIG. 3 depicts the representative results of a transfection experiment using vectors of the invention containing preselected modifications (SEQ ID NOS: 6-10).

FIG. 4 illustrates a Bae 1/U1 construct of the invention. The recognition site of the Bae 1enzyme is underlined, and the 28 base pair fragment which is excised in both strands is shown in bold. In addition, Bae 1 cleaves an additional five nucleotides 3′ of the bold region in one strand, and an additional five nucleotides 5′ of the bold region in the other strand. These sites are indicated with slashes.

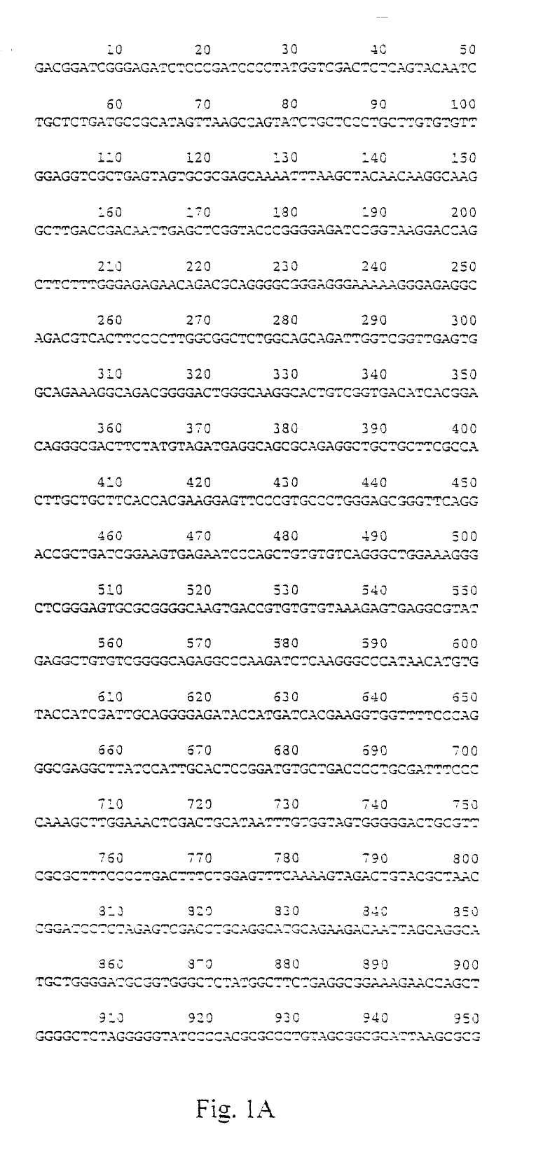

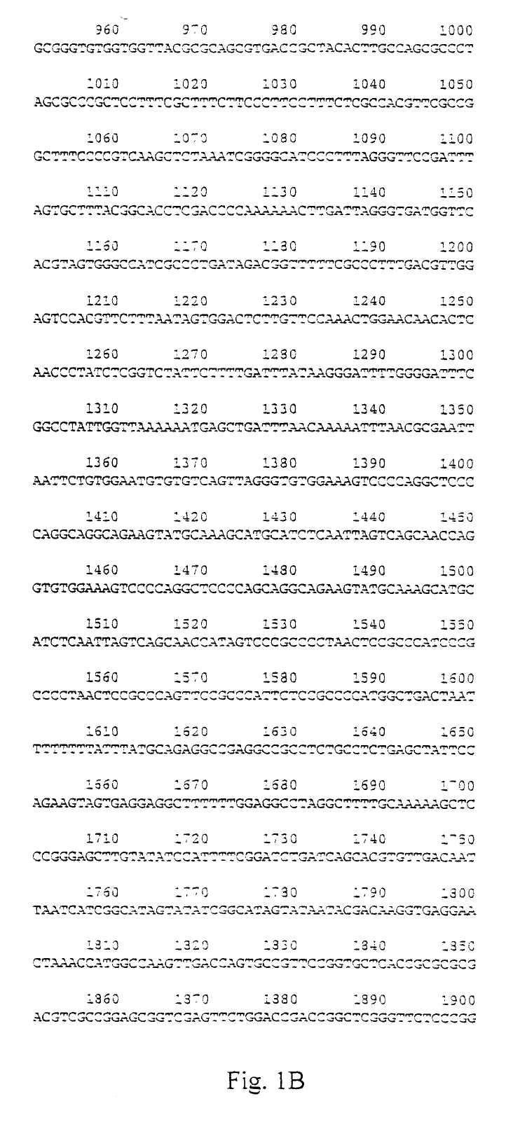

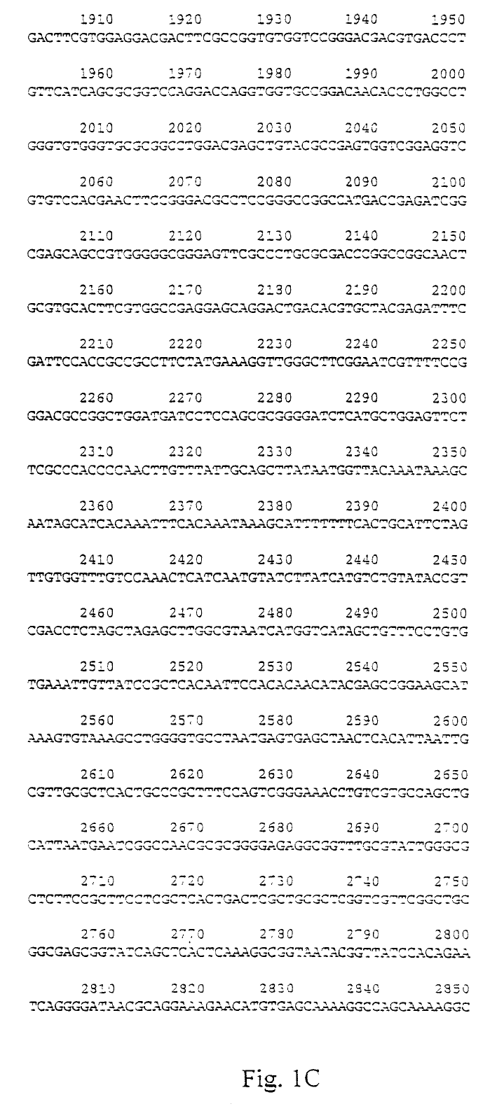

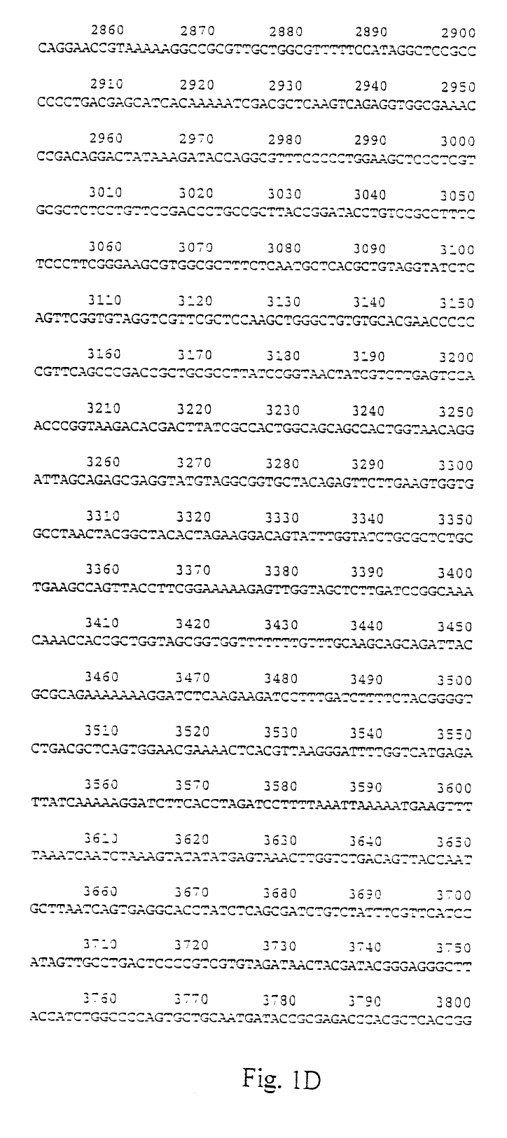

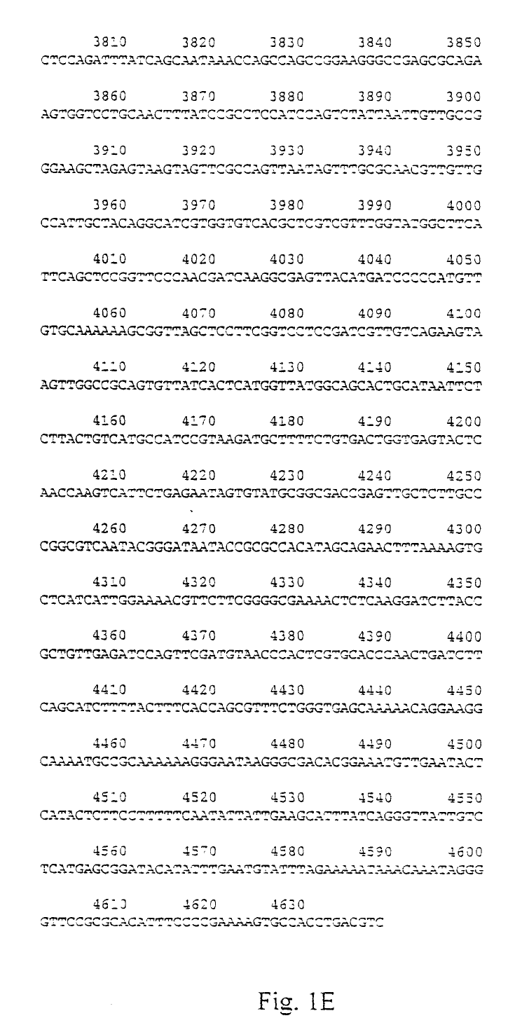

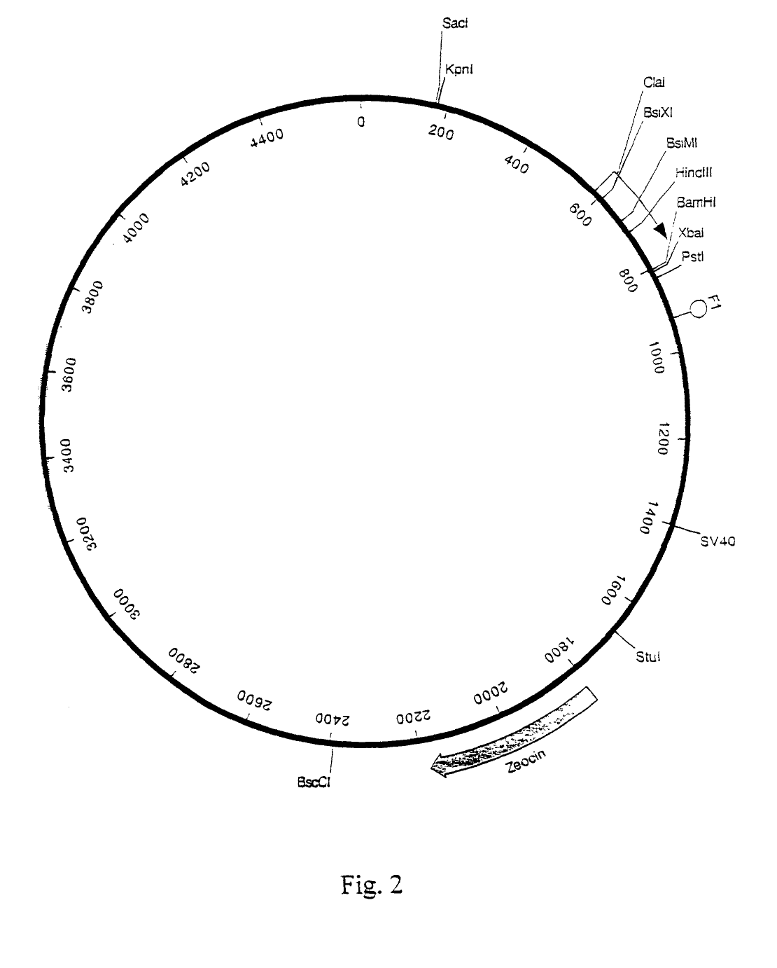

### DETAILED DESCRIPTION OF THE INVENTION

A description of preferred embodiments of the invention follows. It will be understood that the particular embodiments are shown by way of illustration and not as limitations. The principle features of this invention can be employed in various embodiments without departing from the scope of the invention.

Unless otherwise specified, the language and the laboratory procedures used herein relating to cell culture, molecular biology and nucleic acid chemistry are those well known and commonly employed in the art. Standard techniques are used for recombinant nucleic acid methods, nucleotide synthesis, cell culture and transfection. Such techniques and procedures are performed according to conventional methods in the art and as described in various general references well known to those in the art. In general, enzymatic reactions, oligonucleotide synthesis and purification steps performed with commercially supplied products are performed according to the manufacturer's instructions. All patents, applications and references cited herein are incorporated by reference in their entirety.

As described in further detail herein, the invention relates to a recombinant vector comprising an isolated DNA sequence encoding an snRNA (e.g., U1), wherein the snRNA sequence has been modified to contain one or more restriction sites such that digestion with at least one restriction enzyme, and preferably only one restriction enzyme (e.g., Bae 1), allows easy insertion of target-specific sequences (inserts). In a preferred embodiment, the modification is such that the restriction enzyrne(s) cleaves 3′ and 5′ of the region to be excised, thereby eliminating the problems associated with the insertion of additional (extraneous) nucleotides into the snRNA sequence. One advantage of such a vector is more efficient and faster cloning of the inserts, as well as the generation of libraries of snRNA molecules with altered specificity. Vectors of the invention target MRNA comprising a nucleotide sequence which is complementary to the target-specific sequence, thereby inhibiting splicing of the target mRNA and inhibiting expression of the transcription product of the mRNA. Alternatively, vectors of the invention can be used to deliver particular antisense sequences to a target mRNA, thereby inhibiting expression of the transcription product of the mRNA in a manner similar to traditional antisense methodologies.

Recombinant vectors of the invention can be formed by combining the vector components into a vector using conventional methods. The language “vector” is intended to include any molecule which can transport genetic material into an organism while retaining the ability to replicate. Vectors of the invention can be comprised of viral DNA or plasmid DNA. Preferred vectors include retroviral vectors and a variety of plasmids. The language “plasmid” is intended to include any plasmid suitable for use in DNA cloning. Plasmids which are episomal, e.g., capable of extrachromosomal replication can be used, as can plasmids which integrate into the host genome upon introduction. Preferred plasmids are small circular DNA molecules. An example of a suitable plasmid is one based on the pUC18 vector. A particularly preferred plasmid is pcDNA3.1Zeo+.

A vector of the invention can contain isolated DNA which is a full-length or a portion of a wild type DNA sequence encoding a product of interest. The language “wild type” is intended to include a sequence identical to a sequence found in nature. For example, a DNA sequence that is present in an organism is considered to be a wild type sequence. When that sequence has been isolated or reproduced in any manner without intentional modification, the isolated or reproduced sequence is also considered to be wild type. Wild type sequences can include DNA sequences, RNA sequences or amino acid sequences. The language “DNA” is intended to include deoxyribonucleic acid sequences of any size, the language “RNA” is intended to include ribonucleic acid sequences of any size, and the language “amino acid” is intended to include protein and peptide sequences of any size.

Vectors in which the wild type DNA sequence encoding the product of interest has been intentionally modified are included within the scope of the invention. The language “modified” and its variations, such as “modifications”, are intended to include any preselected modification to the wild type DNA sequence encoding a product of interest. The modifications can be contained in an insertion cassette placed within the insertion sites of the vector. In one embodiment, the modification is an alteration of the wild type sequence of the DNA encoding the product of interest. Single nucleotide modifications, e.g., a substitution of one nucleotide of the wild type sequence for a different nucleotide, a deletion of one nucleotide of the wild type sequence or the addition of one nucleotide to the wild type sequence are included. The language also includes modifications to more than one nucleotide, e.g., modifications to a plurality of nucleotides. Such modifications can affect a series of nucleotides adjacent to one another, e.g., contiguous nucleotides. Alternatively, such changes can affect nucleotides not adjacent to one another, e.g., non-contiguous nucleotides. Modifications to a single wild type sequence can also include any variety or combination of modifications to nucleotides contained in any portion of the sequence of the product of interest.

A preferred alteration modification is one which does not unduly affect the function of the expressed product. Particularly preferred alterations are those which contain the same number of nucleotides as contained in the sequence of an excised restriction fragment of the wild type DNA or no more than 1, 2, 3, 4, 5, 6, 7, 8, 9, 10, 15, 20, 25, 30, 40, 50 or 60 additional nucleotides.

In another embodiment, the modification is an insertion of a sequence into the wild type sequence of an isolated DNA encoding a product of interest. In this embodiment, the inserted sequence can include any sequence, whether or not related to the sequence of the DNA encoding the product of interest contained within the vector. The inserted sequence can be an oligonucleotide sequence, e.g., an antisense targeting sequence. The language “antisense” is intended to include a DNA or an RNA sequence that is complementary to at least a portion of a specific mRNA molecule. While not wishing to be bound by theory, it is thought that in the cell, antisense nucleic acids hybridize to a corresponding mRNA, forming a double-stranded molecule. The antisense nucleic acids interfere with the translation of the mRNA, since the cell will not translate an mRNA that is double-stranded.

An insertion modification can be of any size. An insertion modification can be contained in a insertion cassette that replaces an excised restriction fragment of the wild type DNA encoding the product of interest. As such, the insertion modification can contain the same number of nucleotides as contained in the excised restriction fragment, fewer nucleotides than contained in the excised fragment, or more nucleotides than contained in the excised fragment. A preferred insertion modification is one which does not unduly affect the function of the expressed product. Particularly preferred insertions are those which contain the same number of nucleotides as contained in the sequence of an excised fragment of the wild type DNA or no more than 1, 2, 3, 4, 5, 6, 7, 8, 9, 10, 15, 20, 25, 30, 40, 50 or 60 additional nucleotides.

In a preferred embodiment, the vector includes isolated DNA encoding a snRNA. The language “snRNA” is intended to include any small nuclear ribonucleic acid. Preferred snRNAs of the invention are those designated as U snRNAs, e.g., U1 , U2, U3, U4, U5, U6, U7, U8, U9, U10, U11 or U12. Particularlypreferred snRNAs are those which exhibit a splicing function, e.g., U1 or U6. In addition, preferred U snRNAs exhibit a common structural motif termed an Sm site and can also include a 5′ cap structure, such as a trimethylguanosine cap. Without being bound by theory, these structures are thought to promote the nuclear import of the snRNAs and to enhance their stability. Preferably, the isolated DNA sequences encoding the snRNAs are included within the backbone or framework of the vectors of the invention.

In preferred embodiments, vectors containing isolated DNA encoding the U1 snRNA contain a modification within the first 11 nucleotides of the coding sequence. In particularly preferred embodiments, the modifications to this portion of the sequence do not result in the placement of additional nucleotides into the sequence. Modifications to this portion of the U1 snRNA sequence are particularly advantageous because it is the primary area controlling the splicing function of the U1 snRNA. In addition, because U snRNAs are such small molecules, the addition of even a few nucleotides can significantly impact the function of the molecule. Thus, it is particularly desirable to modify the first 11 nucleotides of the U1 snRNA sequence without placing additional nucleotides into the sequence.

The modifications contained within the vectors of the invention, both those described as alteration modifications and those described as insertion modifications, are contained within a modification fragment which is a component of an insertion cassette. The language “modification fragment” is intended to include a double-stranded segment of DNA containing a preselected modification. This fragment can be produced according to methods generally known in the art. The fragment can be isolated from a natural source. The fragment can be synthesized using a commercial apparatus. Often, fragments will be produced as two single strands of DNA, and then allowed to hybridize into a double-stranded fragment.

A modification fragment, in addition to containing a double-stranded DNA encoding a preselected modification, can also be linked to additional single-stranded DNA overhangs. The language “overhang” is intended to include a short single strand of DNA, e.g., several nucleotides, located at either the 5′ or the 3′ end of a modification fragment.

The language “insertion cassette” is intended to include the combination of a two-stranded modification fragment and a pair of single-stranded overhangs, a single overhang located at either end of the modification fragment. In an embodiment, one overhang of the pair of overhangs of the insertion cassette is located at the 3′ end of one strand of the modification fragment, and the other overhang is located at the 3′ end of the complementary strand of the modification fragment. In an alternate embodiment, one overhang of the pair of overhangs of the insertion cassette is located at the 5′ end of one strand of the modification fragment, and the other overhang is located at the 5′ end of the complementary strand of the modification fragment. The overhangs can be of any length. In embodiments, the overhangs are comprised of 3, 4, 5, 6, 7, 8, 9, 10, 15 or 20 nucleotides. In a preferred embodiment, the overhangs are about 5 nucleotides in length.

In a preferred embodiment, an insertion cassette contains a pair of overhangs comprised of single-stranded DNA sequences that are complementary to the DNA sequences of insertion sites contained in the isolated DNA of a vector into which it is to be inserted. The language “insertion site” is intended to include one of the at least two single-stranded portions of DNA located in the isolated DNA of a vector of the invention. In an embodiment, one of the insertion sites is located at the 3′ end of one strand of the double-stranded DNA of the vector, and the other insertion site is located at the 3′ end of the complementary strand of the double-stranded DNA of the vector. In an alternate embodiment, one of the insertion sites is located at the 5′ end of one strand of the double-stranded DNA of the vector, and the other insertion site is located at the 5′ end of the complementary strand of the double-stranded DNA of the vector. The insertion sites can be of any length. In embodiments, the insertion sites are comprised of 3, 4, 5, 6, 7, 8, 9, 10, 15 or 20 nucleotides. In a preferred embodiment, the insertion sites are about 5 nucleotides in length.

Vectors of the invention can include components in addition to the isolated DNA encoding a product of interest. For example, vectors can contain regulatory sequences operably linked to the DNA encoding a product of interest. The language “operably linked” is intended to include the finctional connection between the regulatory sequence and the DNA encoding the transcript. The operably linked regulatory sequence controls the expression of a transcription product. The regulatory sequence may be homologous or heterologous in relation to the encoding DNA. The regulatory sequence can include a promoter. A variety of promoters can be used. A preferred promoter for any particular splicesome snRNA is a wild type promotor generally associated in nature with the DNA encoding the particular splicesome snRNA.

The vectors of the invention can also contain one or more selectable markers, such as those which select for resistance to an antibiotic. Such markers allow for post-transfectional selection of host cells which contain the vector. Alternatively, selectable markers can be contained on another vector which is co-transfected into a host cell. Examples of suitable selectable markers include hprt, neomycin resistance, thymidine kinase, hygromycin resistance, as well as many others known to those of skill in the art.

In general, the recombinant vectors of the invention can be produced by combining the various components using methods known to those of skill in the art. The recombinant vectors of the invention can be produced by inserting isolated DNA encoding a product of interest into a suitable vector. The selection of a suitable vector is determined by a number of factors which include a consideration of the DNA being transfected, the type of host cell being utilized and the type of selectable markers being used. Those of skill in the art can readily determine an optimum vector for any particular use.

The isolated DNA contained in the vector can be contacted with a restriction enzyme. Preferred restriction enzymes of the invention are “dual cleavage” restriction enzymes. The language “dual cleavage” is intended to include restriction enzymes which cleave the isolated DNA of the vector twice. In addition, preferred restriction enzymes cleave DNA and form a restriction fragment which contains a segment of double-stranded DNA linked to two single-stranded DNA overhangs located at each end of the double-stranded segment.

Moreover, particularly preferred restriction enzymes cleave the isolated DNA in two locations outside the recognition site, e.g., once upstream from the recognition site, and once downstream from the recognition site, e.g., once 5′ of the recognition site, and once 3′ of the recognition site. The language “recognition site” is intended to include a short sequence recognized by a particular restriction enzyme or restriction endonuclease. Therefore, when the restriction fragment formed by the restriction enzyme is excised from the isolated DNA of the vector, two corresponding single-stranded insertion sites are formed in the isolated DNA of the vector. The DNA sequences of these insertion sites are complementary to the DNA sequences of the overhangs of the restriction fragment excised from the isolated DNA. In a preferred embodiment, the overhangs have the DNA sequence of 5′-GCAGG-3′ (SEQ ID NO: 2) and 5′-TGAGA-3′ (SEQ ID NO: 3). Moreover, the excision of the restriction fragment can be accomplished without modifying the sequence of the recognition site.

An insertion cassette comprising a double-stranded modification fragment containing a preselected modification linked to two single-stranded overhangs located at each end of the modification fragment can be constructed. The DNA sequences of the overhangs are complementary to the DNA sequences of the insertion sites formed in the isolated DNA upon excision of the restriction fragment, thus permitting the insertion cassette to be readily ligated into the insertion sites of the vector. The insertion cassette, or its components, can be mixed in vitro in the appropriate ratio with a vector and then joined to the vector.

For example, a pUC18 vector is a suitable choice for use as a vector of the invention. The vector can contain a wild type U1 snRNA operably linked to a wild type U1 snRNA promoter. A recognition site for the Bae 1 restriction enzyme, which can be represented as 5′ . . . \hat{0}₁₀(N) A C N N N N G T A Py C (N)₁₂\hat{0} . . . 3′ (SEQ ID NO: 4) and 3′ . . . \hat{0}₁₅(N) T G N N N N C A T Pu (N)₇\hat{0} . . . 5′ (SEQ ID NO: 5) (see New England Biolabs), can be introduced into the U1 sequence contained in the vector. An example of a sequence encoding a U1 snRNA is contained in SEQ ID NO: 1. When the U1 sequence is contacted with the Bae 1 restriction enzyme, the enzyme will first locate its recognition site, and then cleave the DNA sequence twice, once upstream of the recognition site, and once downstream of the recognition site. The restriction fragment formed by this dual cleavage includes a double-stranded DNA segment linked to a pair of single-stranded DNA overhangs at each end of the double-stranded fragment. Because the single-stranded overhangs have two different sequences, the two insertion sites formed in the remaining U1 DNA sequence, which are complementary to the DNA sequences of the overhangs, also have different sequences. Thus, in a rapid and efficient procedure, an insertion cassette containing a preselected modification of the U1 snRNA sequence and a pair of overhangs with single-stranded DNA sequences complementary to the insertion sites formed in the DNA of the vector can be inserted into the vector. Moreover, a Hind III site can be inserted into the vector, for example, in the area of the third loop of the U1 sequence, to assist in the identification of the transfected vectors.

A pcDNA3.1Zeo+ vector is a preferred choice for use as a vector of the invention. Not only does this vector allow for selection of transformants, but it is particularly suited for transfection into host cells of human origin. As previously described with reference to pUC18, this vector can also contain a wild type U1 snRNA operably linked to a wild type U1 snRNA promoter. The recognition site for the Bae 1 restriction enzyme can be introduced into the U1 sequence contained in the vector. Various insertion cassettes containing preselected modifications to the U1 snRNA sequence can be inserted after the vector is digested with the Bae 1 restriction enzyme, and the restriction fragment is excised.

The methods described, which allow modifications to be made to sequences without adding nucleotides, permit certain modifications to be incorporated into the vectors of the invention which were not previously possible. For example, prior to this invention, it was not possible to modify the first 11 nucleotides of the U1 snRNA using a restriction enzyme without adding nucleotides. Since the addition of nucleotides to this portion of the U1 snRNA sequence interferes with its splicing function, effective modification of this portion of the U1 snRNA with a restriction enzyme was not feasible prior to the present invention. Likewise, the ability to introduce a modification without adding additional nucleotides to the sequence is important for all U snRNAs, because they are such small sequences. As described previously, U snRNAs have an average length of only about 250 nucleotides.

To make a determination regarding whether the total number of nucleotides in the isolated DNA of a vector has been altered, a comparison of the number of nucleotides contained in an insertion cassette can be made to the number of nucleotides contained in the restriction fragment excised from the isolated DNA by the dual cleavage restriction enzyme utilized to form the insertion sites. Insertion cassettes which contain a greater number of nucleotides than the comparable restriction fragment result in an increase to the total number of nucleotides in the isolated DNA, while insertion cassettes which contain a smaller number of nucleotides than the comparable restriction fragment result in a decrease in the total number of nucleotides in the isolated DNA. Insertion cassettes which contain the same number of nucleotides as the comparable restriction fragment result in an isolated DNA with the same number of nucleotides as that of the original snRNA sequence.

Although other dual cleavage restriction enzymes can be used in the methods of the invention, the restriction endonuclease described as Bae I is a particularly preferred restriction enzyme. Not only does this enzyme cleave a DNA substrate twice, generating both the requisite restriction fragment and suitable overhangs, but it does so some distance away from its recognition site. The recognition site for Bae I is 5′ . . . \hat{0}₁₀(N) A C N N N N G T A Py C(N)₁₂\hat{0} . . . 3′ (SEQ ID NO: 4) and 3′ . . . \hat{0}₁₅(N) T G N N N N C A T Pu G (N)₇\hat{0} . . . 5′ (SEQ ID NO: 5) (see New England Biolabs) and its cleavage sites are 5′GCAGG-3′ (SEQ ID NO: 2) and 5′-TGAGA-3′ (SEQ ID NO: 3). Therefore, when Bae 1 cleaves the isolated DNA of the vector upstream and downstream from its recognition site, overhangs with two different DNA sequences are formed. Thus, problems associated with recircularization of the plasmid prior to ligation of the desired modification are minimized in the vectors of the invention.

A vector of the invention can be used to transform a host cell. Vectors can optionally, replicate and/or integrate into a recombinant host cell, by known methods. Examples of suitable methods of transfecting or transforming cells include calcium phosphate precipitation, electroporation, microinjection, infection, lipofection and direct uptake. The language “cell” is intended to include not only a particular cell but the progeny or potential progeny of such a cell. Because modifications can occur in succeeding generations, such progeny are characterized by the presence of the transfected vectors, yet they may not be otherwise identical to the subject cell, however, such cells are still included within the scope of the language as it is used herein.

Cells may be prokaryotic or eukaryotic, including plant, fungal, insect and mammalian cells. Preferred cells are mammalian, and can be associated with any mammal of interest. Examples include primates, horses, cows, pigs, rabbits, sheep, dogs and cats. Particularly preferred cells are human. Both primary and immortalized cells can be used.

Methods for preparing such recombinant host cells are described in more detail in Sambrook et al., “Molecular Cloning: A Laboratory Manual,” Second Edition (1989) and Ausubel, et al., “Current Protocols in Molecular Biology,” (1992), for example. After preparation, recombinant cells can be cultured under suitable conditions. Generally, the cells are maintained in a suitable buffer and/or growth medium or nutrient source for growth of the cells and expression of the gene product. The growth media are not critical to the invention, are generally known in the art and include sources of carbon, nitrogen and sulfur. Examples include Dulbecco's modified eagles media (DMEM), RPMI-1640, M199 and Grace's insect media.

An optimal pH is selected based on the requirements of the host cell, and the cell is maintained under suitable temperature and atmospheric conditions. Temperature is also preferably selected based on the particular requirements of the host cell and can be, for example, between about 35° C. and 40° C. Products produced by the host cells can be isolated and purified by known methods.

A library of host cells can be obtained by transfecting suitable host cells with vectors of the invention. For example, a number of vectors can be used to create a library of host cells containing a variety of snRNA modifications. The host cells can be of any type including bacterial, yeast and mammalian, including human cell types. For embodiments related to libraries of snRNA modifications, host cells of human origin are preferred. The cell library can be used to propagate and amplify the vectors containing the modifications until sufficient numbers of the modifications can be obtained. Proper transfection of a large percentage of the modifications is assured by the directional nature of the insertion cassette, e.g. the appropriate overhangs will ligate to the appropriate insertion sites. Moreover, problems associated with self-closure of the plasmid are minimized. Clones exhibiting a desired phenotype can be identified by any of the methods known in the art and permitted by the particular construction of the vector, e.g., by use of the incorporated selectable marker. Selected clones can be tested to determine the functional nature of the sequence in the construct. Thus, novel DNA having specific functions can be identified

The vectors of the invention can also be used in a method of identifying a modification of a snRNA which suppresses transcription of a transcription product in a cell. A base level of transcription of a transcription product in a cell can be determined. A variety of recombinant vectors comprising isolated DNA encoding a snRNA in which the isolated DNA of each of the recombinant vectors comprises an insertion cassette containing a different modification fragment contained between at least two insertion sites of the vector can be produced. Any number of modifications can be produced due to the rapid and efficient methods provided by the invention. In embodiments, 5, 10, 15, 20, 25, 50, 75, 100 and 200 or more vectors containing different and unique modifications can be produced. Each vector containing a modification can be introduced into a cell, under conditions suitable for delivery of the snRNA into the cell. Then, the level of transcription of the transcription product in each cell containing a vector containing the modified snRNA can be compared with the base level of transcription of the transcription product in the cell, and a determination can be made regarding which snRNA modifications inhibit transcription in the cell. In general, if the level of transcription of the transcription product in the cell including the vector containing the modified snRNA is less than the base level of transcription of the transcription product in the cell, a modification which suppress expression of a transcription product in the cell has been identified.

The vectors of the invention can also be used to create transgenic animals by introducing DNA sequences into the germ line cells of non-human animals. Methods for producing transgenic animals through the use of embryo manipulation and microinjection are well known in the art. For example, Hogan, 1986, Manipulating the Mouse Embryo (Cold Spring Harbor Laboratory Press, Cold Spring Harbor, N.Y.), describes methods for generating transgenic mice. Such methods can also be used to generate other species of transgenic animals.

The vectors of the invention can be utilized to suppress the expression of a transcription product in a cell. For example, a vector of the invention can be utilized to deliver a modified U1 snRNA to a cell. A U1 snRNA, for example, modified to interfere with the splicing process of a particular DNA so that the reading frame is not preserved during intron removal, can result in the suppression of the transcription product in that cell.

Furthermore, the vectors of the invention can also be utilized to deliver antisense targeting sequences into cells. Under suitable conditions, the antisense targeting sequences can hybridize to the corresponding sequences forming a double-stranded molecule which inhibits translation. The inhibition of translation prevents the production of the transcription product in the cell.

The invention is further illustrated by the following non-limiting example.

### EXAMPLE

Material and Methods

Vector construction

The U1/Bae 1 vector was digested overnight using Bae 1. The vector was purified using qiaquick spin columns. For each ligation, two oligos were designed with complementary sequences. The forward primer and the reverse primer contain 5 extra bases at the 3′ end (5′-GCAGG-3′ (SEQ ID NO: 2) and 5′-TGAGA-3′ (SEQ ID NO: 3), respectively). Primers were diluted to 20 μM stock, and 2 μL of each stock was combined in 21 μL dH20. Primers were heated to 95° C. for 1 minute, then allowed to anneal at 70° C. for 10 minutes. Subsequently, the sample was allowed to cool to room temperature.

4 μL of the annealed primers were combined with 100 ng of the digested U1/Bae 1 vector and ligated using the Boehringer Mannheim Rapid DNAL ligation kit, according to manufacturer's recommendation. XL-10 gold bacterial cells (Stratagene, La Jolla, Calif.) were transformed with 2 μL of the ligation mixture and plated on 1.5% agarose LB plates, in the presence of 50 μg/mL ampicillin.

From each plate, 10 colonies were picked at random and used to inoculate a standard PCR reaction using two U1 gene specific primers. Following PCR, 2.5 units of Cla1 were added to the PCR reaction. Clones which resulted in a 700 bp PCR product which was not digested by Cla1 were sequenced to confirm the correct sequence of the insert. All cultures were purified using qiagen midi columns before transfection.

Cell culture and transfection

293T cells were cultured according to standard conditions. The night before transfection, cells were counted and plated at 1×10⁶ cells per well in 6 well plates. A particular U1/Bae 1 construct and a luciferase encoding plasmid, pSP-luc+, (Clontech, Palo Alto, Calif.) were combined in a 3:1 molar ratio. Cells were transfected using Lipofectamine Plus (LIFE Technologies, Rockville, Md.) according to the manufacturer's recommendation. Assays for luciferase activity and protein content were performed 48-72 hours after transfection. All samples were transfected in triplicate.

Luciferase assay

Cells were lysed using RIPA buffer (Roche, Indianapolis, IN). Protein concentration was then determined using the Bio-Rad protein assay kit and luciferase activity was determined using Steady-Glo Luciferase assay system (Promega Corp., Madison, Wis.). Luciferase activity was normalized to protein content and all values were normalized to the level of cells co-transfected with a wild type U1 sequence.

Results

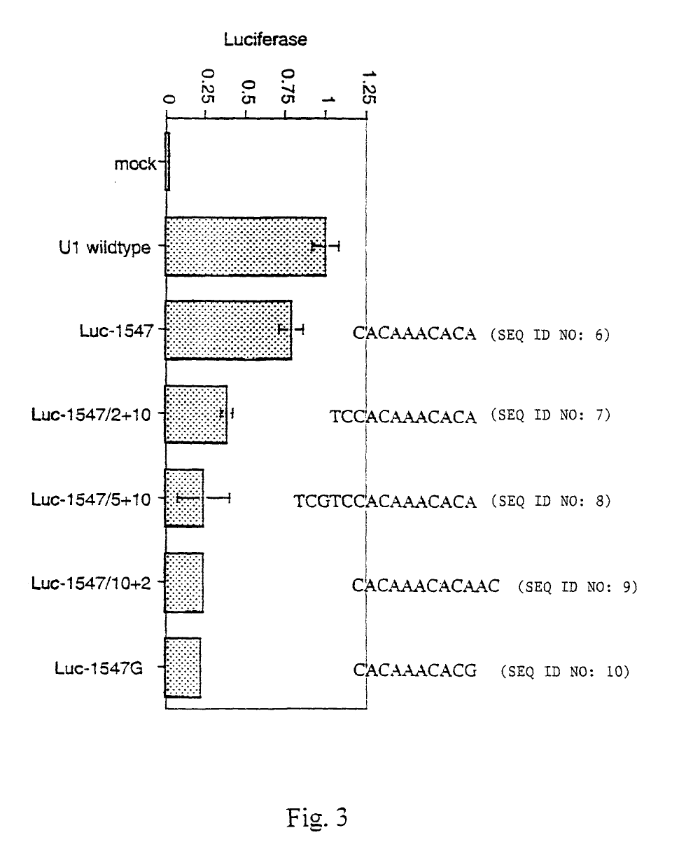

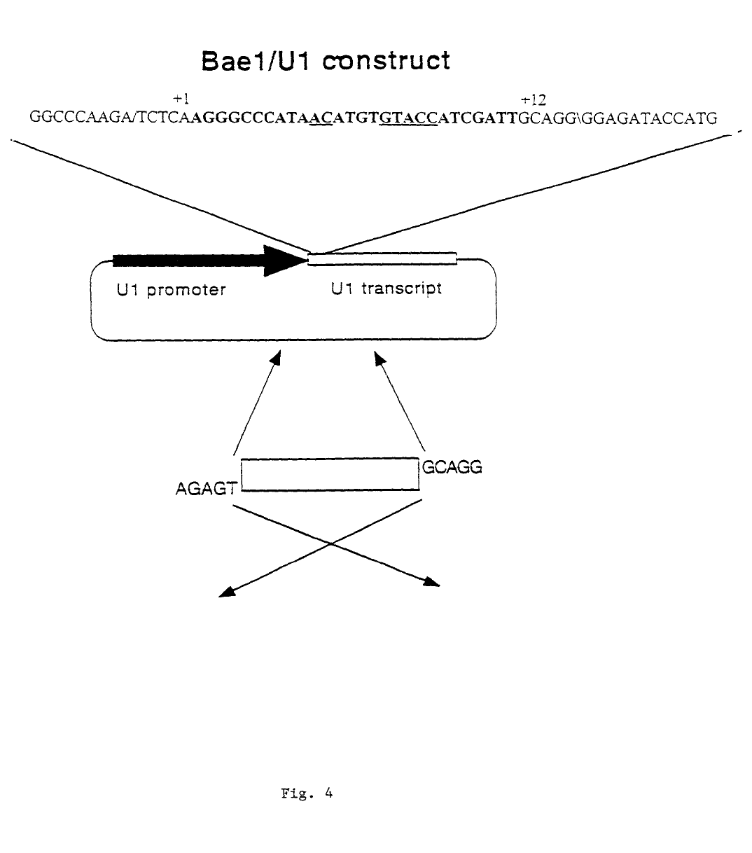

A sequence directed against bases 1547-1556 of luciferase was cloned into the U1/Bae 1 vector. This construct, Luc-1547 (SEQ ID NO: 6), was co-transfected with luciferase and gave a consistent downregulation of luciferase activity by approximately 20% (FIG. 3). In order to increase the effect, the sequence was extended in the 3′ direction to encompass 12 or 15 bases, Luc-1547/2+10 (SEQ ID NO: 7), Luc-1547/5+10 (SEQ ID NO: 8), respectively, of the luciferase target sequence. The Luc-1547/2+10 and Luc 1547/5+10 constructs were more active against co-transfected luciferase with a clear increase in activity with an increase in size (FIG. 3). A construct with an extension of the target sequence by 2 bases in the 5′ direction, Luc-1547/10+2 (SEQ ID NO: 9), led to an even more dramatic effect on co-transfected luciferase activity (FIG. 3).

Careful analysis of the original sequence revealed the potential disturbance of a stem-loop structure present in the original U1 sequence through base pairing with the 3′-most A in the cloned 10 base sequence of Luc-1547, Luc-1547/2+10 and Luc-1547/5+10. When the 3′-most A was replaced with a G and the resulting construct, Luc-1547G (SEQ ID NO: 10), transfected, it was indeed more active in downregulation of co-transfected luciferase (FIG. 3).

### EQUIVALENTS

While this invention has been particularly shown and described with reference to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed by the scope of the claims.

What is claimed is:

1. A recombinant vector comprising isolated DNA encoding a snRNA, wherein the isolated DNA comprises an insertion cassette contained between at least two insertion sites.

2. The vector of claim 1, wherein the snRNA is selected from the group of snRNAs with splicing functions.

3. The vector of claim 2, wherein the snRA is U1 snRNA.

4. The vector of claim 2, wherein the snRNA is U6 snRNA.

5. The vector of claim 3, wherein the insertion cassette comprises a modification fragment of about 30 base pairs of DNA.

6. The vector of claim 5, wherein the modification fragment contains a modification to the first 11 nucleotides of a U1 snRNA.

7. The vector of claim 6, wherein a single nucleotide is modified.

8. The vector of claim 6, wherein a plurality of nucleotides are modified.

9. A recombinant vector comprising isolated DNA encoding a snRNA, wherein the isolated DNA comprises a Bae 1 restriction fragment.

10. The vector of claim 9, wherein the isolated DNA further comprises two insertion sites formed by the excision of the Bae 1 restriction fragment.

11. The vector of claim 10, wherein the two insertion sites comprise the complements of DNA sequences of SEQ ID NO: 2 and SEQ ID NO: 3.

12. The vector of claim 10, further comprising an insertion cassette.

13. The vector of claim 12, wherein the insertion cassette contains the same number of nucleotides as contained in the Bae 1 restriction fragment.

14. The vector of claim 12, wherein the insertion cassette contains more nucleotides than were contained in the Bae 1 restriction fragment.

15. A method of producing a recombinant vector comprising isolated DNA encoding a product of interest, wherein the isolated DNA comprises an insertion cassette contained between at least two insertion sites comprising the following steps:
(a) inserting isolated DNA encoding a product of interest into the vector;
(b) contacting the isolated DNA with a dual cleavage restriction enzyme that excises a restriction fragment comprising a double-stranded DNA modification fragment linked to a single-stranded DNA overhang at each end of the modification fragment;
(c) excising the restriction fragment from the isolated DNA of the vector such that at least two insertion sites are formed in the isolated DNA;
(d) obtaining an insertion cassette wherein the single-stranded overhang at each end of the modification fragment comprises a DNA sequence complementary to the DNA sequence of the insertion sites formed in the isolated DNA; and
(e) ligating the insertion cassette into the isolated DNA of the vector between the insertion sites formed by the excision of the restriction fragment,
thereby producing a recombinant vector comprising an insertion cassette contained between at least two insertion sites.

16. The method of claim 15, wherein the product of interest is selected from the group of snRNAs with splicing functions.

17. The method of claim 15, wherein the snRNA is U1 snRNA.

18. The method of claim 15, wherein the snRNA is U6 snRNA.

19. The method of claim 17, wherein the insertion cassette comprises a modification fragment of about 30 base pairs of DNA.

20. The method of claim 19, wherein the insertion cassette contains a modification to the first 11 nucleotides of a U1 snRNA.

21. The method of claim 20, wherein a single nucleotide is modified.

22. The method of claim 20, wherein a plurality of nucleotides are modified.

23. The method of claim 17, wherein the dual cleavage restriction enzyme is Bae 1.

24. The method of claim 17, wherein each insertion site consists of about 5 nucleotides.

25. A cell transformed by a recombinant vector comprising isolated DNA encoding a snRNA, wherein the DNA comprises an insertion cassette contained between at least two insertion sites.

26. The cell of claim 25, wherein the cell is a mammalian cell.

27. A cell library comprising cells transformed by a plurality of recombinant vectors comprising isolated DNA encoding a snRNA, wherein the DNA comprises an insertion cassette contained between at least two insertion sites.

28. The cell library of claim 27 comprising insertion cassettes containing at least about 10 different modification fragments.

29. A method of identifying a modification of a snRNA which suppresses transcription of a transcription product in a cell comprising:
(a) determining a base level of transcription of a transcription product in a cell;
(b) producing at least 10 recombinant vectors comprising isolated DNA encoding a snRNA, wherein the isolated DNA of each of the recombinant vectors comprises an insertion cassette containing a different modification fragment contained between at least two insertion sites of the vector;
(c) introducing each vector containing a modification into a cell, under conditions suitable for delivery of the snRNA into the cell;
(d) comparing the level of transcription of the transcription product in each cell containing a vector containing the modified snRNA with the base level of transcription of the transcription product in the cell; and
(e) determining which snRNA modifications inhibit transcription in the cell,
whereby, if the level of transcription of the transcription product in the cell containing the vector containing the modified snRNA is less than the base level of transcription of the transcription product in the cell, a modification which suppress expression of a transcription product in the cell has been identified.

30. A method of suppressing expression of a transcription product in a cell comprising:
(a) producing a recombinant vector comprising isolated DNA encoding a snRNA, wherein the isolated DNA comprises an insertion cassette contained between at least two insertion sites;
(b) introducing the vector into the cell, under conditions suitable for delivery of the snRNA into the cell; and
(c) utilizing the snRNA to inhibit transcription in the cell, thereby, suppressing expression of a transcription product in the cell.

31. A method of delivering an antisense targeting sequence into a cell nucleus comprising:
(a) inserting an antisense targeting sequence into a recombinant vector comprising an isolated DNA encoding a snRNA, wherein the DNA comprises an insertion cassette contained between at least two insertion sites; and
(b) introducing the vector into the cell, under conditions suitable for delivery of the antisense targeting sequence across the cell membrane and into the cell nucleus,
whereby the antisense targeting sequence is delivered to the cell nucleus.
